(19) United States
(12) Patent Application Publication
(10) Pub. No.: US 2013/0226021 A1
(43) Pub. Date: Aug. 29, 2013

(54) PROTECTIVE ENSEMBLE HYDRATION MONITOR

(71) Applicant: Albert K. Dec, Deerfield, NH (US)

(72) Inventor: Albert K. Dec, Deerfield, NH (US)

(73) Assignee: BAE Systems Information and Electronics Systems Integrations Inc., Nashua, NH (US)

(21) Appl. No.: 13/879,549

(22) PCT Filed: Aug. 9, 2012
(86) PCT No.: PCT/US12/50048

§ 371 Date: Apr. 15, 2013

Related U.S. Application Data
(60) Provisional application No. 61/522,725, filed on Aug. 12, 2011.

(51) Int. Cl. A61B 5/00

(52) U.S. Cl. CPC A61B 5/4875
USPC 600/532

(57) ABSTRACT

A protective ensemble hydration monitor configured to be disposed within a face mask of a protective ensemble is disclosed. In one embodiment, the protective ensemble hydration monitor includes a humidity sensor to detect the user's breath within the face mask and output a breath signal based on the detected user's breath. Further, the protective ensemble hydration monitor includes a microprocessor coupled to the humidity sensor to receive the breath signal and output a control signal based on a pre-determined humidity threshold value. Furthermore, the protective ensemble hydration monitor includes a vibrator configured to provide a tactile feedback to the user upon receiving the control signal from the microprocessor.

### CROSS-REFERENCE TO RELATED APPLICATION

This Application claims rights under 35 USC §119(c) from U.S. Application 61/522,725 filed Aug. 12, 2011, the contents of which are incorporated herein by reference.

### BACKGROUND OF THE INVENTION

1. Field of the Invention

The present invention relates to a protective ensemble, more specifically to a protective ensemble including a protective ensemble hydration monitor.

2. Brief Description of Related Art

Typically, soldiers become dehydrated easily when they are in a protective gear, such as a mission oriented protective posture (MOPP) gear, because the soldiers do not breathe and the soldiers tend to sweat a lot. There are provisions to take in water for drinking while in the protective gear and the soldiers are trained to hydrate continuously, however, the soldiers tend to get busy and get distracted, therefore, dehydration is a big problem when the soldiers are in such protective gear.

There have been many studies conducted on effectiveness of the soldiers when they get dehydrated, in addition to health risks and lost time due to treatment, they tend to start losing command and control as they can get disoriented and unable to make decisions. Therefore, keeping the soldiers hydrated when they are wearing such protective gear is of great importance.

It can be envisioned that similar dehydration problem exist with fighter pilots wearing oxygen masks and underwater scuba divers wearing a scuba gear/suit.

### SUMMARY OF THE INVENTION

A protective ensemble hydration monitor is disclosed. According to one aspect of the present subject matter, a protective ensemble for protecting a user from harmful environmental substances includes a face mask and the protective ensemble hydration monitor designed to be disposed within the face mask and configured to detect humidity of the user's breath and notify the user based on a pre-determined humidity threshold value.

According to another aspect of the present subject matter, the protective ensemble hydration monitor includes a humidity sensor, a microprocessor coupled to the humidity sensor, a power source coupled to the humidity sensor, and a pressure sensor, a vibrator and memory coupled to the micro processor. Further, the protective ensemble hydration monitor includes a communication link coupled to the microprocessor.

In operation, the pressure sensor provides a pressure control signal to activate the protective ensemble hydration monitor upon detecting a predetermined over pressure within the face mask. Further, the humidity sensor detects the user's breath within the face mask and output a breath signal based on the detected user's breath. Furthermore, the microprocessor receives the breath signal and outputs a control signal, via the communication link, based on the pre-determined humidity threshold value stored in the memory. In addition, the vibrator provides a tactile feedback to the user upon receiving the control signal from the microprocessor.

### BRIEF DESCRIPTION OF THE DRAWINGS

The advantages and features of the present disclosure will become better understood with reference to the following detailed, description and claims taken in conjunction with the accompanying drawings, wherein like elements are identified with like symbols, and in which:

FIG. 1 illustrates a protective ensemble including a protective ensemble hydration monitor, according to an embodiment of the present subject matter;

FIG. 2 illustrates the protective ensemble hydration monitor, such as shown in FIG. 1, including major components suitable for implementing some aspects of the present subject matter; and

FIG. 3 illustrates a block diagram of the protective ensemble hydration monitor, such as shown in FIGS. 1 and 2, providing monitoring signals to a medical service facility, according to an embodiment of the present subject matter.

### DETAILED DESCRIPTION OF THE INVENTION

The exemplary embodiments described herein in detail for illustrative purposes are subject to many variations in structure and design.

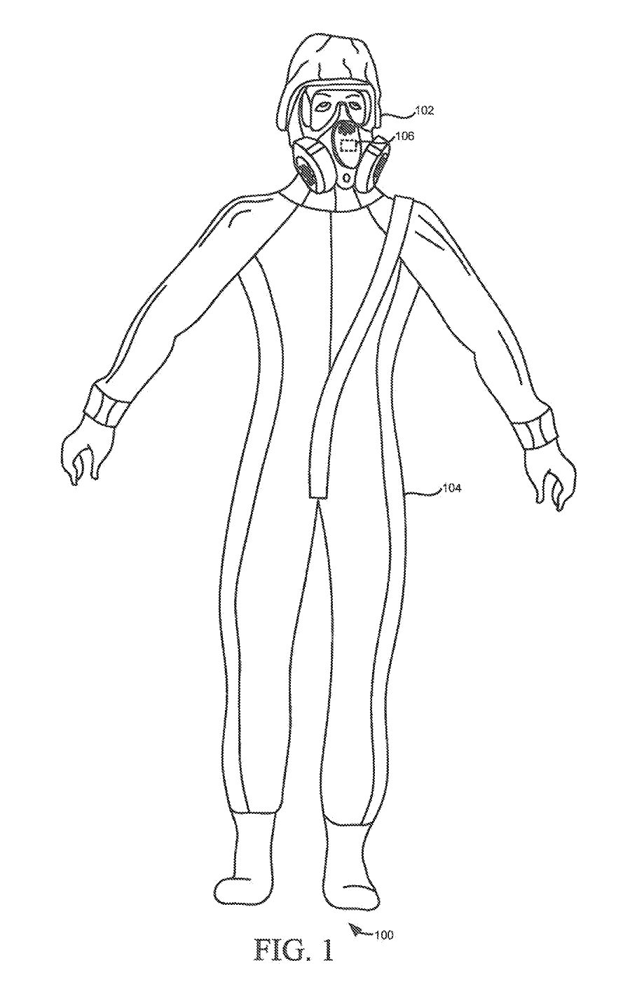

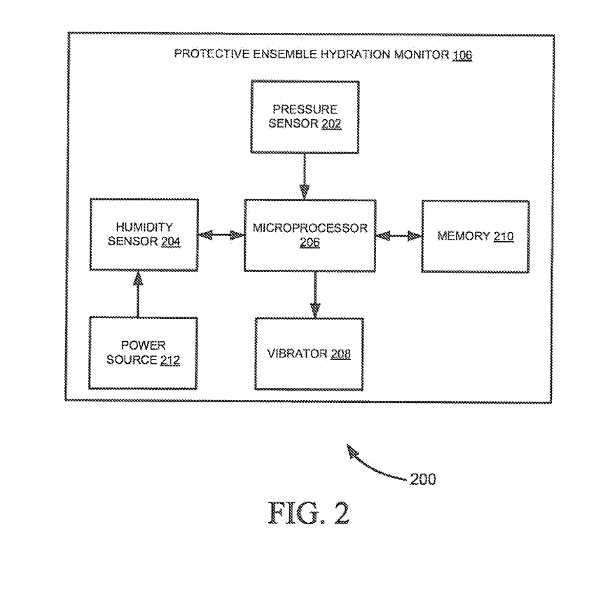

FIG. 1 illustrates a protective ensemble 100 including a protective ensemble hydration monitor 106, according to an embodiment of the present subject matter. For example, the protective ensemble 100 includes a mission oriented protective posture (MOPP) gear, a fighter pilot oxygen mask gear, a firefighter scott air pack system, an underwater scuba diving gear and the like. As shown in FIG. 1, the protective ensemble 100 includes a face mask 102 and a protective garment 104. Further, the face mask 102 includes the protective ensemble hydration monitor 106. Furthermore, the protective garment 104 is connected to the face mask 102. In operation, the protective ensemble hydration monitor 106 detects humidity of the user's breath and notifies the user based on a pre-determined humidity threshold value. This is explained in more detail with reference to FIG. 2.

Referring now to FIG. 2, which is the protective ensemble hydration monitor 106, such as shown in FIG. 1, including major components suitable for implementing some aspects of the present subject matter. As shown in FIG. 2, the protective ensemble hydration monitor 106 includes a pressure sensor 202, a humidity sensor 204, a microprocessor 206, a vibrator 208, memory 210, and a power source 242. The power source 212 is configured to power the humidity sensor 204. Exemplary power source 212 includes a battery and the like. For example, the vibrator 208 includes a piezoelectric vibrator, a buzzer and the like. Further, the microprocessor 206 and the power source 212 are coupled to the humidity sensor 204, Furthermore, the microprocessor 206 is coupled to the vibrator 208. Also, the pressure sensor 202 the memory 210 are coupled to the microprocessor 206.

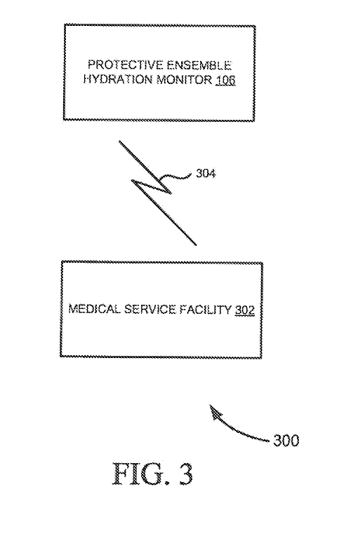

In operation, the pressure sensor 202 provides a pressure control signal activate the protective ensemble hydration monitor 106 upon detecting a predetermined over pressure within a face mask, such as shown in FIG. 1. Further, the humidity sensor 204 detects the user's breath within the face mask and output a breath signal based on the detected user's breath. Furthermore, the microprocessor 206 receives the breath signal and outputs a control signal, via the communication link, based on the pre-determined humidity threshold value stored in the memory 210. For example, the microprocessor 206 outputs the control signal when a humidity level of the user's breath is below the pre-determined humidity threshold value. In one embodiment, the microprocessor outputs monitoring signals to a medical service facility, via the communication link, based on the pre-determined humidity threshold value. This is explained in more detail with reference o FIG. 3. In addition, the vibrator 208 provides a tactile feedback to the use upon receiving the control signal from the microprocessor 206. In one embodiment, the vibrator 208 provides buzzing sensation to the user upon receiving the control signal from the microprocessor 206.

Referring now to FIG. 3, which is a block diagram 300 that illustrates the protective ensemble hydration monitor 106, such as shown in FIGS. 1 and 2, providing monitoring signals to a medical service facility 302, according to an embodiment of the present subject matter. A shown in FIG. 3, the block diagram 300 includes the protective ensemble hydration monitor 106 and the medical service facility 302. Further, the protective ensemble hydration monitor 106 includes a communication link 304. In one embodiment, the communication link 304 is coupled to a microprocessor, such as shown in FIG. 2. Exemplary communication link includes a radio frequency (RF) link. In operation, the protective ensemble hydration monitor 106 detects the user's breath within a face mask, such as shown in FIG. 1, and provides the monitoring signals to the medical service facility 302 via the communication link 304. In one embodiment, the protective ensemble hydration monitor 106 provides status of the user's hydration level to the medical service facility 302 via the communication link 304.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

We claim:

1. A protective ensemble for protecting a user from harmful environmental substances, comprising:
a face mask; and
a protective ensemble hydration monitor designed to be disposed within the face mask and configured to detect humidity of the user's breath and notify the user based on a pre-determined humidity threshold value.

2. The protective ensemble of claim 1, wherein the protective ensemble hydration monitor comprises:
a humidity sensor to detect the user's breath within the face mask and output a breath signal based on the detected user's breath;
a microprocessor coupled to the humidity sensor to receive the breath signal and output a control signal based on the pre-determined humidity threshold value; and
a vibrator configured to provide a tactile feedback to the user upon receiving the control signal from the microprocessor.

3. The protective ensemble of claim 2, wherein the protective ensemble hydration monitor further comprises:
a power source; and
memory to store the pre-determined humidity threshold value.

4. The protective ensemble of claim 3, wherein the power source is a battery.

5. The protective ensemble of claim 2, wherein the protective ensemble hydration monitor further comprises a communication link coupled to the microprocessor to output the control signal, wherein the communication link comprises a radio frequency (RF) link.

6. The protective ensemble of claim 2, wherein the protective ensemble hydration monitor further comprises a pressure sensor coupled to the microprocessor and configured to provide a pressure control signal to activate the protective ensemble hydration monitor upon detecting a predetermined over pressure within the face mask.

7. The protective ensemble of claim 1, wherein the protective ensemble is selected from the group consisting of a mission oriented protective posture (MOPP) gear, a fighter pilot oxygen mask gear a firefighter scott air pack system, and an underwater scuba diving gear.

8. A protective ensemble hydration monitor designed to be disposed within a face mask of a protective ensemble, comprising:
a humidity sensor to detect the user's breath within the face mask and output a breath signal based on the detected user's breath;
a microprocessor coupled to the humidity sensor to receive the breath signal and output a control signal based on a pre-determined humidity threshold value; and
a vibrator configured to provide a tactile feedback to the user upon receiving the control signal from the microprocessor.

9. The protective ensemble hydration monitor of claim 8, further comprising:
a power source; and
memory to store the pre-determined humidity threshold value.

10. The protective ensemble hydration monitor of claim 9, wherein the power source is a Battery.

11. The protective ensemble hydration monitor of claim 8, further comprising:
a communication link coupled to the microprocessor to output the control signal, wherein the communication link comprises a radio frequency (RF) link.

12. The protective ensemble hydration monitor of claim 8, wherein the protective ensemble hydration monitor further comprises a pressure sensor coupled to the microprocessor and configured to provide a pressure control signal to activate the protective ensemble hydration monitor upon detecting a predetermined over pressure within the face mask.
